(19) United States
(12) Patent Application Publication
(10) Pub. No.: US 2009/0197797 A1
(43) Pub. Date: Aug. 6, 2009

(54) USE OF HYALURONIC ACID AS A CARRIER MOLECULE FOR DIFFERENT CLASSES OF THERAPEUTIC ACTIVE AGENTS

(75) Inventors: Stefano Norbedo, Trieste (IT); Susanna Bosi, Monfalcone (IT); Massimo Bergamin, Trieste (IT); Riaz Ahmed Khan, Berkshire (GB); Erminio Murano, Trieste (IT)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: EURAND PHARMACEUTICALS LTD., Bray, Co. Wicklow (IE)

(21) Appl. No.: 12/162,337

(22) PCT Filed: Jan. 25, 2007
(86) PCT No.: PCT/EP2007/050726

§ 371 Date: Jul. 25, 2008

(30) Foreign Application Priority Data
Jan. 25, 2006 (IE) 2006/0049

(51) Int. Cl. A61K 38/38; C07H 5/06; A61K 31/728

(52) U.S. Cl. 514/8; 536/55.2; 514/54

(57) ABSTRACT

The present invention refers to a drug delivery system consisting of hyaluronic acid and a therapeutic active agent.

### PRIOR ART

Many drugs, which are hydrophobic in character and hence show poor solubility in water have been conjugated with hydrophilic polymers to increase their water solubility and improve the bioavailability. For this purpose a number polymeric materials showing the property of biocompatibility, biodegradability have been used, some of them are bioactive, have sufficient drug loading capacity, and have drug targeting capabilities. Examples are polyglutamate, polyethylene glycole, carboxymethyl dextran and hyaluronic acid. However, PLG, PEG and CMD lack in bioactivity and targeting capabilities while HA has the advantage over the others because in addition it is bioactive and has the capability to target the drug to the diseased site. Many tumour types overexpress CD44 receptors; and HA can be used to conjugate anticancer drugs to target the delivery of the drug to the diseased site. Endocytosis of derivatised HA has been shown in cell lines expressing CD44 HA receptor. The fluorescent labelled HA-Taxol conjugate has been shown to be selectively toxic towards human cancer cell lines which were known to overexpress HA receptors. The presence of liver receptors for HA (HARLEC) suggests that it can be used as a carrier molecule to target a drug to the liver tissue. HA has been demonstrated for liver metastases from a colon adenocarcinoma in mice.

The preparation of HA substituted at the C-6 primary hydroxyl group with dihydrofolate reductase inhibitors (DHFR) have been described in WO0168105. This conjugate has been obtained by preparing HA-6-halogen by selective halogenation reaction of HA, and followed by displacement of the halogen by the DHFR. This conjugate is still endowed with antiproliferative activity, however it still presents the problem that it contains residual halogen groups.

Selective introduction of a leaving group on polysaccharide has been described in Carb. Res. 340, 2229-2235, 2005 where a tosylation of cellulose in a mixture of acetamide and lithium chloride is reported; the conditions chosen allows the complete sulfonylation of all the primary hydroxyl groups with the aim of blocking said positions and introducing other chemical groups on the free positions.

### DESCRIPTION OF THE FIGURES

FIG. 1: represents the formula of DDSs: HA-6-methotrexate, HA-6-ibuprofen, HA-6-PenG

FIG. 2: represents the DOSY NMR spectrum of HA-6-OMs obtained in example 9 (in DOSY weighed mono-dimensional NMR spectra only rigid macromolecules are present, furnishing evidence for polymer chemical modification)

FIG. 3: represent the ¹³C NMR spectrum of HA-6-OMs, peaks of salifying DIEA are present.

FIG. 4: represents the DOSY NMR spectrum of HA-6-MTX obtained in example 24

FIG. 5: represents the ¹³C NMR spectrum of HA-6-MTX obtained in example 24

FIG. 6: represents the DOSY NMR spectrum of HA-Ibuprofen obtained in example 26

FIG. 7: represents the ¹³C NMR spectrum of HA-Ibuprofen obtained in example 26

FIG. 8: represents the DOSY NMR spectrum of HA-Penicillin G obtained in example 29

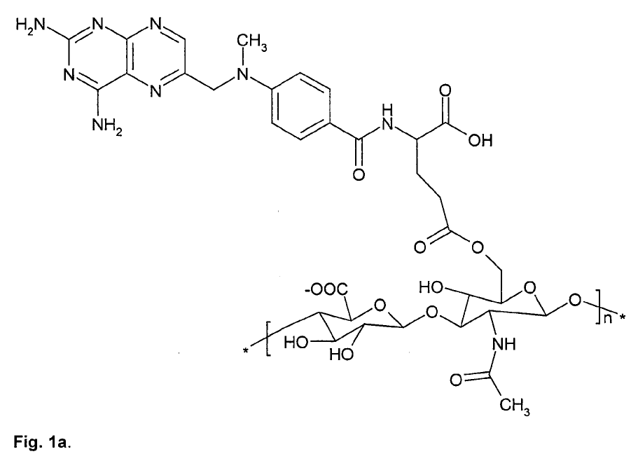

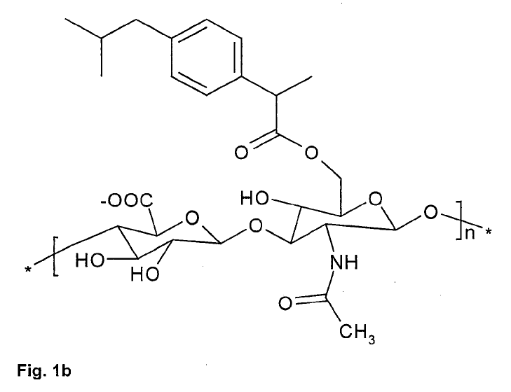

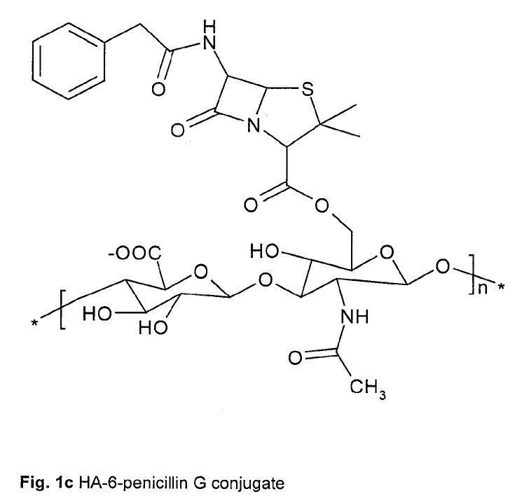

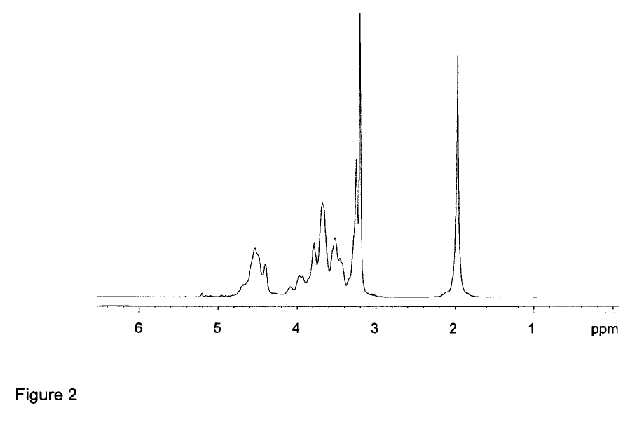

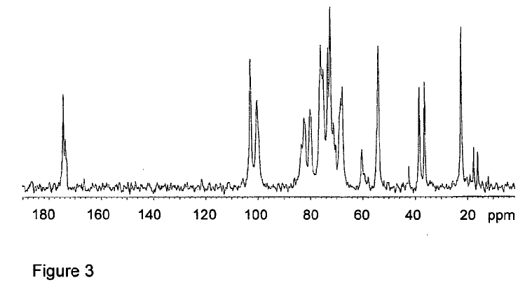

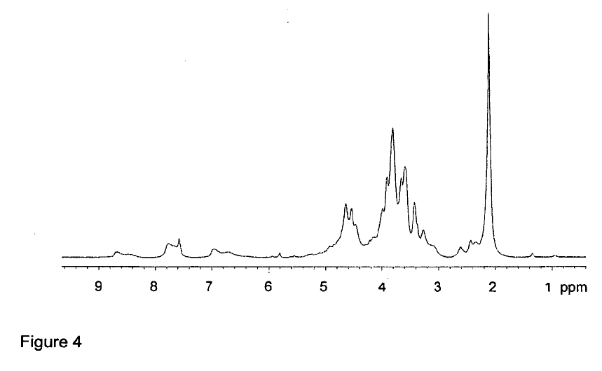

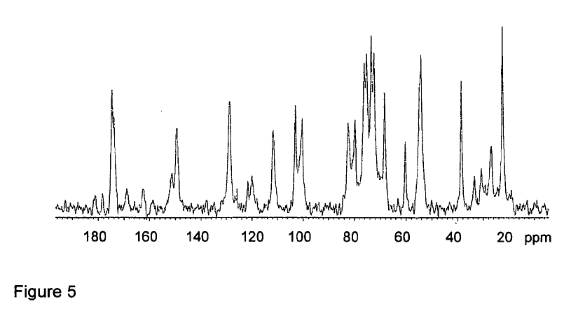

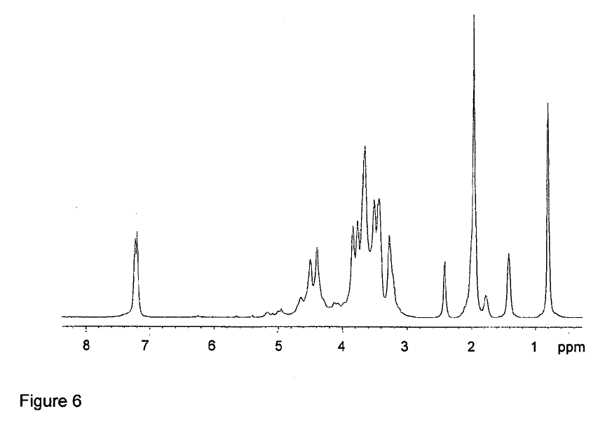

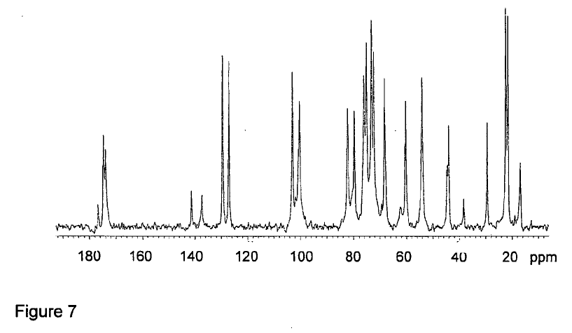

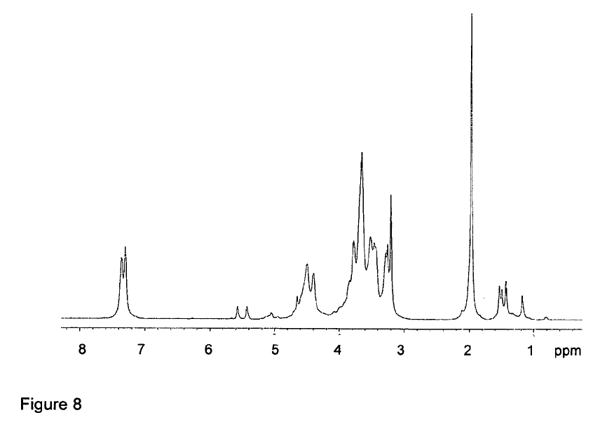

### DETAILED DESCRIPTION OF THE INVENTION

In a first aspect of the invention, there is provided a drug delivery system (DDS) consisting of hyaluronic acid (HA) and a therapeutic active agent, whereby this active agent is covalently linked at the C-6 position of the N-acetyl-D-glucosamine residue of the hyaluronic acid with the exception of active agents of formula (I):

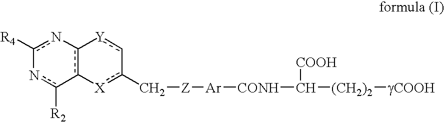

wherein:
R₂ and R₄ independent from one another represent: —NH₂, —OH, —OCH₃, C₁-C₅ alkyl, ═O; X and Y represent: —C(R₅)═, —CH(R₅)—, —NH—, —N═), wherein R₅ represents: —H, C₁-C₅ alkyl; Z represents: —CH(R₁₀)—, —N(R₁₀)—, —O—; R₁₀ represents: —H, C₁-C₅ alkyl, C₁-C₅ alkenyl, C₁-C₅ alkynyl, 5-6 membered heterocyclic ring with 1-3 heteroatoms selected in the group consisting of nitrogen, sulphur and oxygen; Ar represents: 1,4-phenyl group, 1,4-phenyl group condensed with one or more 5-6 membered aromatic rings, 1,4-phenyl group condensed with one or more 5-6 membered heterocycles, wherein said Ar is possibly substituted with R₂; rings A and b, independently from one another, may be aromatic or non-aromatic.

The compounds of formula (I) are the dihydrofolato reductase inhibitors described in WO0168105.

Hyaluronic acid (also herein indicated as HA) is composed of a disaccharidic repeating unit, consisting of D-glucuronic acid and 2-acetamido-2-deoxy-D-glucose (N-acetyl-D-glucosamine) bound by β(1→3) glycosidic linkage; the D-glucuronic acid residue may either be in the acid form or in the form of a salt. Each repeating unit is bound to the next one by a β(1→4) glycosidic linkage that forms a linear polymer.

The term hyaluronic acid, as used in the present invention, encompasses both the acid and the salified form.

The term hyaluronic acid is commonly used to describe a general group of molecular fractions of HA with varying molecular weights or also hydrolysed fractions of said compound. For the purposes of the present invention the hyaluronic acid has preferably an average molecular weight comprised between 10000 to 1 million and more preferably 20000 to 500000.

The therapeutic active agent is chosen from drugs belonging to a number of different therapeutic categories: analgesic, antihypertensive, anestetic, diuretic, bronchodilator, calcium channel blocker, cholinergic, CNS agent, estrogen, immunomodulator, immunosuppressant, lipotropic, anxiolytic, antiulcerative, antiarrhytmic, antianginal, antibiotic, anti-inflammatory, antiviral, thrombolitic, vasodilator, antipyretic, antidepressant, antipsychotic, antitumour, mucolytic, narcotic antagonist, hormones, anticonvulsant, antihistaminic, antifungal, antipsoriatic.

These therapeutic active agents contain a nucleophilic group. A nucleophilic group is an electron-pair donor group such as carboxylic, amino, substituted amino, hydroxyl, thiol, amide group; the carboxylic group is preferred.

In the DDS the linkage between the hyaluronic acid and the active agent is an ester, an amino, an ether, a thioether, an amide. The ester linkage is preferred.

The DDSs are either in the acid form or in the salt form. When they are in salt form they may be salified with alkaline metals (preferably Na or K), earth-alkaline metals (preferably Ca or Mg), transition metals (preferably Cu, Zn, Ag, Au, Co, Ag). The salification is obtained by processes known by the skilled artisan.

Optionally, also the secondary hydroxyl groups on the DDSs may be derivatised to form a group selected from: —OR, —OCOR, —SO₂H, —OPO₃H₂, —O—CO—(CH₂)ₙ—COOH, —O—(CH₂)ₙ—OCOR, wherein n is 1-4 and R is C₁-C₁₀ alkyl, —NH₂, —NHCOCH₃. These substitutions can be easily obtained by processes known in the art, and they may be chosen in order to modulate the hydrophilic character of the DDSs.

The total amount of the therapeutic active agent in the DDSs is defined by the degree of substitution (C6-DS); the latter can alternatively indicate the % by weight of the active agent with respect to the total weight of the DDS (C6-DS_{w}) or the % by mole of the active agent with respect to the mole of repeating unit of modified HA (C6-DSₘₒₗ).

In the DDS of the invention the C6-DS_{w} is preferably comprised between 0.1 and 60%, more preferably between 1 and 50%, even more preferably between 5 and 40%.

As demonstrated in the experimental part, the invented DDSs are characterised by the presence of active agent directly linked to the primary hydroxyl groups of the N-acetyl-D-glucosamine units of the hyaluronic acid. No other hydroxyl groups of the HA are involved in the chemical linkage with the drug. Moreover, the DDSs are stable and free of undesired reaction by-products and impurities that can be harmful to their practical pharmaceutical use.

They retain the pharmaceutical effect of the therapeutic agent. Therefore, they can be successfully used in the treatment of all pathologies that are appropriate for the specific therapeutic active agent in the DDS.

Accordingly, it is a further aspect of the invention the use of the above DDSs in the manufacture of a medicament for the treatment of pathologies appropriate for each therapeutic agent. Said pathologies are selected from the group consisting of tumours, skin disorders, psoriasis, inflammatory pathologies, rheumatoid arthritis, and infectious diseases.

It is also an aspect of the invention a pharmaceutical composition containing the DDSs of the invention in admixture with pharmaceutically acceptable excipients and/or diluents. The pharmaceutical composition may be either in the liquid or in solid form; it may be administered through the oral, parenteral, topical route. Particularly interesting are the injectable pharmaceutical compositions containing the invented DDSs.

A further aspect of the invention is a technology for the preparation of the drug delivery system of HA and a therapeutic active agent with the exception of compounds of formula (I) having the features described above. It has been surprisingly found that the reaction does not only occurs with compound having the structure of formula (I) having two carboxylic groups and heterocyclic rings, but this process is widely applicable to a high number of different active agents which belong to different therapeutic categories.

This technology comprises the following reaction steps:

(a) introducing a leaving group at the C-6 position of the N-acetyl-D-glucosamine units of the hyaluronic acid either in the free form or in the salt form thus obtaining a HA-6-activated
(b) forming a chemical linkage between the C6 position of the HA-6-activated and the therapeutic active agent by displacing the leaving group (at the C6 position of HA) with a nucleophilic group present on the therapeutic active agent, thereby obtaining a HA-6-active agent
(c) possible displacing of any un-substituted leaving group from the HA-6-active agent obtained in step (b)
(d) recovering the HA-6-active agent

With this process it is possible to obtain DDSs having a C6-DS_{w} preferably comprised between 0.1 and 60%, more preferably between 1 and 50%, and even more preferably between 5 and 40%.

There are two different ways of carrying out the process of the invention. In a first way the HA-6-activated obtained from step (a) is isolated from the reaction mixture and then reacted with the therapeutic active agent according to step (b) to give the final HA-6-active agent that may optionally undergo step (c).

In the second way of carrying out the process, the step (b) is performed directly on the reaction mixture obtained in step (a) that contains the HA-6-activated. The advantage of this second way of performing the reaction consists in the fact that the isolation step of the HA-6-activated is avoided.

The starting HA may be in free form or in the form of salt, wherein the counterion is preferably an alkaline or alkaline-earth metal or is a nitrogen-containing counterion. In the latter case the counterion may contain heterocycles selected from the group consisting of pyridine, pyrazine, pyrimidine, pyrrole, pyrazole, imidazole triazole, tetrazole, possibly substituted with one or more C₁-C₆ alkyl groups. Preferred examples of nitrogen-containing counterions are ammonium, tetrabutylammonium (TBA), pyridinium or sym-collidinium ions.

Step (a) is a selective reaction carried out by adding the suitable reagent to a thoroughly stirred suspension or solution of HA (in free form or in the salified form) in an aprotic organic solvent.

The leaving group which is introduced at the C-6 position of the glucosamine unit of the HA is any electron-pair acceptor group that departs during the substitution by a nucleophile group. It may be selected from the group consisting of sulfonate group, phosphonate group (triphenylphoshonate), cyanide (CN—), nitrite (NO2-), halogen (preferably chloro), sulphate group, halogensulfate group, nitrate, halogensulfite (chlorosulfite).

When the leaving group is halogen the halogenation is carried out as described in WO9918133 and WO0168105. Among the halogen group the chlorine group is the preferred one and the preferred reagent to perform the halogenation is methanesulfonyl chloride in N,N-dimethylformamide.

This step allows the formation of the HA-6-activated.

Step (b) is performed by reacting the hyaluronic acid-6-activated or one of its salt obtained form step (a) with the therapeutic active agent. It consists in the substitution of the leaving group by the nucleophilic group contained in the active agent and entails the formation of a covalent linkage between the C-6 position of hA and the active agent. The chemical nature of said linkage depends on the chemical nature of nucleophile group. It may be an ester linkage which is formed when the nucleophile is a carboxylic group. Other linkages that are formed between the HA and the therapeutic active agent are: amino, ether, thioether, amide.

Step (c) is a possible step that may be any suitable reaction that allows the displacement of any possible un-substituted leaving group. Such a displacement may be carried out for example by photolyisis, by reduction. In some case, step (c) is not necessary since some un-substituted leaving group may be destroyed during the step (b) either because of the reaction conditions or during the work-up.

In step (d) the obtained the HA-6-active agent (DDS) is recovered by means of standard techniques.

In a preferred embodiment of the process the leaving group is the sulfonyl group and the obtained activated HA is therefore HA-6-sulfonated. This preferred reaction comprises the following reaction steps:

(a) introducing a sulfonate group at the C-6 position of the N-acetyl-D-glucosamine units of the hyaluronic acid in the salt form thus obtaining a HA-6-sulfonated
(b) forming a chemical linkage between the C-6 position of the HA-6-sulfonated and the therapeutic active agent by displacing the sulfonated group (at the C-6 position of HA) with the nucleophilic group present on the therapeutic active agent, thereby obtaining a HA-6-active agent.
(d) recovering the HA-6-active agent

In this embodiment, the selective sulfonylation reaction of step (a) is carried out using as sulfonylating reagent an alkyl- or aryl-sulfonyl halide, preferably chloride, in presence of an organic or inorganic base, preferably an organic base. The alkyl- or aryl-sulfonyl halide may be chosen among, preferred are methylsulfonyl (mesyl), toluene-p-sulfonyl (tosyl), trifyl, trimsyl, tripsyl, 1,1-sulfonyl-imidazole.

The organic base is selected preferably among the different organic amines, such as diisopropylethylamine, triethylamine.

The solvent is chosen from the group consisting of: dimethylformamide, dimethylacetamide, dimethylsulfoxide, formamide.

The general sulfonylation procedure is as follows. The base, preferably organic base is added to a suspension or a solution of HA in salt form, preferably in an organic base form, by stirring under nitrogen flux. Then the alkyl- or aryl-sulfonyl chloride in a suitable solvent, preferably the same solvent, is added dropwise. After a period of time ranging from 2 to 90 minutes (preferably 45-75 min), the reaction is quenched by addition of NaHCO₃ to remove the formate ester groups formed during the reaction at secondary hydroxyl groups of HA. Then the reaction is allowed to continue for about 10-20 hours, preferably 18 hours. The reaction product (HA-6-sulfonated) is either directly recovered form the solution by means of known techniques, such as precipitation, drying or before recovery the solution is treated in such a way as to allow the obtainment of the HA-6-sulfonated in a suitable salt form, such as HA-6-sulfonated:TBA.

The reaction conditions are mild; in fact, reaction can be successfully carried out at room temperature or at a lower temperature, no cooling-heating cycles are required, pH conditions are mild.

The reagent is used in limited quantities, the suitable amount is 1-10 molar equivalents with respect to the repeating HA unit (preferably 2-6 molar eq) of sulfonyl halide (such as mesylchloride), in the presence of 2-20 molar equivalents with respect to the repeating HA unit (preferably 4-12 molar eq) of organic amine (such as DIEA).

Under the above reaction conditions the obtained hyaluronic acid-6-sulfonated has degree of substitution (DSₘₒₗ), ranging from 10% to 91% mol/mol, preferably from 20 to 90%, even more preferably from 40 to 80%. The selectivity of the mesylation reaction for the primary position (C-6) of the N-acetyl-D-glucosamine residue is between 50 and 100% (C6-DSₘₒₗ). Some mesylation reactions also occurs at the secondary positions, such as at C-4 of N-acetyl-D-glucosamine and at the C-2, C-3 positions of the D-glucuronic acid residue. Their structures and the degree of mesyl group substitution in the polymer are confirmed by NMR spectroscopy.

In a preferred embodiment of the sulfonylation reaction, step (b) entails the formation of an ester linkages group between the HA and the carboxylic group present on the therapeutic agent.

In this last embodiment, step (a) is carried out as described above and step (b) is usually performed according to the following procedure.

A solution of the carboxylic group containing-active agent is added to a solution of the HA-6-sulfonated either in TBA or in the sodium salt form, preferably TBA, in presence of an alkaline or alkaline-earth metal salt, such as cesium carbonate. The reaction is carried out between 40-90° C., preferably 80° C. under constant stirring, preferably under nitrogen flux for a period of time ranging form 5 to 42 hours, preferably form 8 to 20 hours (18 hours). The reaction mixture is worked up according to known techniques.

A further aspect of the present invention is a drug delivery system consisting of hyaluronic acid and a compound of formula (I), whereby the carboxylic group of compound of formula (I) is covalently linked at the C-6 position of the N-acetyl-D-glucosamine units of the hyaluronic acid by means of an ester linkage and said DDS is obtained by the specific process described hereunder. These new DDSs contain the compound of formula (I) directly linked at the C-6 position of the HA and are characterised by the fact and no other hydroxyl groups of the HA repeating unit is involved in chemical linkage neither with the drug nor with other chemical groups. In particular these DDSs are devoid of any residual leaving groups (such as sulfonate group) both on the primary and on the secondary positions of the HA units. The term “devoid” means that the residual leaving group is present in an amount below 0.5% w/was determined by NMR. These features allows the maintenance of the regularity of the original HA chemical structure and the retention of the configuration of the carbon atoms, these properties/aspects are highly important to ensure the efficacy and the interaction with the specific receptors.

Differently, the conjugate of HA and methotrexate that was described in WO0168105 contains residual chlorine atoms, that are introduced on the polysaccharide during the halogenation step.

Among the different compounds having formula (I) the preferred one is methotrexate. Methotrexate (MTX) is represented by formula (I) where R₂ and R₄ are —NH₂; ring A is aromatic; ring B is aromatic; X and Y are: —N═; Z is: —N(CH₃)—; Ar is: 1,4-phenyl group. The C6-DS_{w} of the DDSs is preferably comprised between 0.1 and 60%, more preferably between 1 and 50%, even more preferably between 5 and 40%, the MW is comprised between 10,000 and 500,000.

The technology for the preparation of this DDS comprises the following reaction steps:

(a) introducing at the C-6 position of the N-acetyl-D-glucosamine units of the hyaluronic acid either in the free form or in the salt form a leaving group selected from the group consisting of sulfonate group, phosphonate group (triphenylphoshonate), cyanide (CN—), nitrite (NO2-), sulphate group, halogensulfate group, nitrate, halogensulfite (chlorosulfite) thus obtaining a HA-6-activated
(b) forming an ester linkage between the C6 position of the HA-6-activated and the compound of formula (I) by displacing the leaving group (at the C6 position of HA) with a carboxylic group present on compound (I), thereby obtaining a HA-6-compound of formula (I)
(d) recovering the HA-6-compound of formula (I)

In the preferred embodiment, step (a) is a sulfonylation reaction and the reagent used for introducing the sulfonate group is an alkyl- or aryl-sulfonyl halide, preferably chloride, in presence of an organic or inorganic base. The preferred the reagent is methylsulfonyl chloride or toluene-p-sulfonyl chloride and the organic base is diisopropylethylamine or triethylamine.

The DDS can be obtained with the above process according to two different ways. In the first way the HA-6-sulfonated obtained from step (a) is isolated from the reaction mixture and then reacted with the compound of formula (I) according to step (b) to give the final HA-6-compound of formula (I).

In the second way of carrying out the process, the step (b) is performed directly on the reaction mixture obtained in step (a) that contains the HA-6-sulfonated. The advantage of this second way of performing the reaction consists in the fact that the isolation step of the HA-6-sulfonated is avoided.

### EXPERIMENTAL PART

### Example 1

### Determination of Structure

The determination of mesylate content in the HA-6-Mesylate (HA-Ms) by NMR was achieved by integration of the peaks in the region 3.10÷3.32 ppm (1H of HA chain and 3H of mesylate) versus the peak at 1.95 ppm (3H of HA chain).

### Example 2

### Determination of Structure

The determination of tosylate content in the HA-6-tosylate (HA-Ts) by NMR was achieved by integration of the peaks of tosylate at 7.8 ppm (2H), 7.5 ppm (2H) and 2.45 ppm (3H) versus the peak at 1.95 ppm (3H of HA chain).

### Example 3

### Determination of Structure

Determination of methotrexate content in HA-6-MTX by NMR was achieved by integration of the peaks in the region 6.0÷8.6 ppm (5H of MTX) versus the peaks in the region 1.85÷2.58 ppm (3H of HA chain and 4H of MTX).

### Example 4

### Determination of Structure

The determination of Ibuprofen in HA-6-Ibuprofen by NMR was achieved by integration of the peaks of ibuprofen in the regions 7.02÷7.24 ppm (4H), 2.38 ppm (2H), 1.40 ppm (3H), 0.78 ppm (6H) versus the peak of the HA chain at 1.95 ppm (3H).

### Example 5

### Determination of Structure

The determination of Penicillin G in HA-6-Penicillin G by NMR was achieved by integration of the peaks of Penicillin G in the regions 7.05÷7.20 ppm (5H), 5.55 ppm (1H), 5.40 (1H) versus the peak of the HA chain at 1.95 ppm (3H).

### Example 6

Methotrexate content by HPLC was determined by analysing the samples before and after alkaline hydrolysis according to Methotrexate Official Monograph (USP 23-p 984). The analyses conditions were: Cromatograph: Dionex DX-600. Column: Column Phenomenex Synergi 4μ Hydro-RP80, Column size: 150×460 mm, Column particle size: 4μ, Temperature: 40° C. Eluent: 90% 0.2M dibasic sodium phosphate/0.1M citric acid (630:270), 10% CH₃CN, isocratic condition: 0.5 mL/min. Detector: Diode Array (range 200-780 nm), Selected wavelength for the quantitative determination: 302 nm Injected volume: 25 μl, run time 30 minutes. Solutions for free methotrexate determination were prepared by dissolving HA-MTX directly in MilliQ water at the appropriate concentration. Total methotrexate content was determined after alkaline hydrolysis carried out in NaOH 0.1 M, room temperature for 2 hours. After neutralization with hydrochloric acid 1 M, solutions were filtered through 0.45 μm (Sartorius Minisart RC25 17795Q) prior to injection in the HPLC system. A calibration curve was determined by using standard solutions with known concentration of methotrexate. The method gives the MTX concentration in the sample solution, which normalized by the sample concentration yields the DS_{weight} % w/w.

### Example 7

### Determination of Weight Average Molecular Weight (Mw)

The molecular weight of the hyaluronic acid DDS was measured by HP-SEC (High Performance Size Exclusion Chromatography). The analysis conditions were: Chromatograph: HPLC pump 980-PU (Jasco Ser. No. B3901325) with Rheodyne 9125 injector. Column: TSK PWxl (TosoBioscience) G6000+G5000+G3000 6, 10, 13 μm particle size; Temperature: 40° C. Mobile phase: NaCl 0.15 M+0.01% NaN₃. Flux: 0.8 mL/min. Detector: MALS (WYATT DAWN EOS—WYATT, USA), λ=690 nm, (dn/dc=0.167 mL/g), UV spectrophotometric detector 875-UV (Jasco, Ser. No. D3693916), λ=305 nm, Interferometric Refractive Index OPTILAB REX (WYATT, USA); 1=690 nm, Sensitivity: 128×; Temperature: 35° C. Injected volume: 100 μl, run time 60 minutes.

The samples of HA-Cl, HA-OMs, HA-MTX, HA-conjugated to different drugs to be analysed were solubilised in 0.9% NaCl at the concentration of about 1.0 mg/ml and kept under stirring for 12 hours. Then, the solutions were filtered on a 0.45 μm porosity filter (Sartorius Minisart RC25 17795Q) and finally injected in the chromatograph. The analysis allows the measurement of Mw (weight average molecular weight), Mn (number average molecular weight), PI (polydispersity). Mw and Mn values are expressed as g/mole. The concentration of the polymeric samples solutions were controlled by means of the integral of the refractive index.

### Example 8

### Preparation of 6-O-Methanesulfonylhyaluronic acid (HA-6-Ms or HA-Ms)

To a solution of 500 mg (0.806 mmol) of TBA salt of HA (MW 20,000) in 20 ml of dimethylsulfoxide (DMSO) were added 1.11 ml (6.48 mmol) of diisopropylethylamine (DIEA) by stirring under nitrogen. Methanesulfonyl chloride (MsCl) (314 μL; 4.03 mmol) was then added dropwise at room temperature, whereupon an orange solution formed. After 1 h stirring at room temperature, one third of the reaction mixture was quenched by pouring into saturated NaHCO₃ solution (50 ml), stirring overnight at pH 9. The resulting solution was ultrafiltered, concentrated in a rotary evaporator and freeze-dried to afford 40 mg of an off-white solid (total DS 83% mol/mol by NMR).

The rest of the reaction mixture was stirred overnight and then worked up as described above, to obtain 90 mg of an off-white solid (total DS 86% mol/mol by NMR).

Overall yield: 130 mg of HA-Ms sodium salt (40%).

¹H NMR (D₂O) ppm: 1.95 (s, 3H, NHCOCH₃), 3.23 (s, 2.58H, MsO), 3.2÷4.2 (m, 7.42H, HA chain), 4.3÷4.7 (m, 2H, anomeric+1.72H, CH—OMs); ¹³C NMR (D₂O) ppm: 23 (NHCOCH₃), 37 (MsO), 55, 61 (CH₂OH), 68, 69.5 (CH₂O Ms), 72, 74, 75, 76, 80, 83, 101, 103, 174, 175.

### Example 9

### Preparation of 6-O-Methanesulfonylhvaluronic acid (HA-Ms)

To a solution of 5.00 g (8.06 mmol) of TBA salt of HA (MW 20,000) in 200 ml of DMSO were added 13.9 ml (81 mmol) of DIEA by stirring under nitrogen. MsCl (3.2 ml; 41 mmol) was then added dropwise at room temperature, whereupon an orange solution was formed. After 1 h stirring at room temperature, the reaction mixture was quenched by pouring into saturated NaHCO₃ solution (400 ml), bringing the total volume to 1 L with water (resulting pH: 9.5) and maintaining stirring overnight. The resulting solution was ultrafiltered under a hood and concentrated in a rotary evaporator. A small portion was evaporated to dryness in a rotary evaporator (100 mg) for NMR analysis: total mesylate DS 91% mol/mol by proton NMR, primary mesylates 58% mol/mol by carbon NMR, selectivity 64% for the C6 position.

The rest of the solution was treated with amberlite IRA-120 loaded with TBA and freeze-dried to afford 4.62 g of an off-white solid (HA-Ms:TBA salt).

### Example 10

### Preparation of 6-O-Methanesulfonylhyaluronic Acid (HA-Ms)

To a solution of 2.50 g (4.03 mmol) of TBA salt of HA (MW 20,000) in 100 ml of DMSO were added 5.6 ml (32.7 mmol) of DIEA by stirring under nitrogen. MsCl (1.3 ml; 16.7 mmol) was then added dropwise at room temperature, whereupon an orange solution was formed. After 1 h stirring at room temperature, the reaction mixture was quenched by pouring into saturated NaHCO₃ solution (200 ml), bringing the total volume to 600 ml with water (resulting pH: 9.2), and maintaining stirring overnight. The resulting solution was ultrafiltered under a hood and concentrated in a rotary evaporator. A small portion was freeze-dried (136 mg) for NMR analysis: total mesylate DS 79% mol/mol by proton NMR, primary mesylates 64% mol/mol by carbon NMR, selectivity 81% for C6 position.

The rest of the solution was treated with amberlite IRA-120 loaded with TBA and freeze-dried to afford 2.10 g of an off-white solid (HA-Ms:TBA salt).

### Example 11

### Preparation of 6-O-Methanesulfonylhyaluronic Acid (HA-Ms)

To a solution of 3.00 g (4.84 mmol) of TBA salt of HA (MW 20,000) in 100 ml of DMSO were added 8.4 ml (48.4 mmol) of DIEA by stirring under nitrogen. MsCl (1.92 ml; 24.2 mmol) was then added dropwise at room temperature, whereupon an orange solution formed. After 15 min stirring at room temperature, the reaction mixture was quenched by pouring into saturated NaHCO₃ solution (200 ml), bringing the total volume to 600 ml with water (resulting pH: 9.5) and maintaining stirring overnight. The resulting solution was ultrafiltered under a hood and concentrated in a rotary evaporator. A small portion was freeze-dried (187 mg) for NMR analysis: total mesylate DS 76% mol/mol by proton NMR, primary mesylates 58% mol/mol by carbon NMR, selectivity 76% for C6.

The rest of the solution was treated with amberlite IRA-120 loaded with TBA and freeze-dried to afford 2.561 g of an off-white solid (HA-Ms:TBA salt).

### Example 12

### Preparation of 6-O-Methanesulfonylhyaluronic acid (HA-Ms)

To a solution of 3.00 g (4.84 mmol) of HA TBA salt (MW 20.000) in DMSO (100 ml) were added 4.96 ml (29.0 mmol) of DIEA by stirring under nitrogen. MsCl (1.13 ml; 14.5 mmol) was then added dropwise at room temperature, whereupon an orange solution was formed. After 15 min stirring at room temperature, the reaction mixture was quenched by pouring into saturated NaHCO₃ solution (200 ml), bringing the total volume to 600 ml with water (resulting pH: 9.5) and maintaining stirring overnight. The resulting solution was ultrafiltered under a hood and concentrated in a rotary evaporator. A small portion was freeze-dried (248 mg) for NMR analysis: total mesylate DS 55% mol/mol by proton NMR, primary mesylates 41% mol/mol by carbon NMR, selectivity 75% for C6.

The rest of the solution was treated with amberlite IRA-120 loaded with TBA and freeze-dried to afford 2.41 g of an off-white solid (HA-Ms:TBA salt).

### Example 13

### Preparation of 6-O-Methanesulfonylhyaluronic Acid (HA-Ms)

To a solution of 3.00 g (4.84 mmol) of HA TBA salt (MW 20.000) in DMSO (100 ml) was added DIEA (4.96 ml; 29.0 mmol) by stirring under nitrogen. MsCl (1.13 ml; 14.5 mmol) in dichloromethane (20 ml) was then added dropwise during 20 min, at room temperature, whereupon an orange solution formed. The reaction mixture was then immediately quenched by pouring into saturated NaHCO₃ solution (200 ml), bringing the total volume to 600 ml with water (resulting pH: 9.5) and maintaining stirring overnight. The resulting solution was ultrafiltered under a hood and concentrated in a rotary evaporator. A small portion was freeze-dried (172 mg) for NMR analysis: total mesylate DS 85% mol/mol by proton NMR, primary mesylates 50% mol/mol by carbon NMR, selectivity 59% for C6.

The rest of the solution was treated with amberlite IRA-120 loaded with TBA and freeze-dried to afford 2.78 g of an off-white solid (HA-Ms:TBA salt).

### Example 14

### Preparation of 6-O-Methanesulfonylhyaluronic Acid (HA-Ms)

To a suspension of 3.00 g (7.48 mmol) of HA sodium salt (MW 20.000) in DMSO (100 ml) were added DIEA (12.8 ml; 74.8 mmol) and MsCl (2.90 ml; 37.4 mmol), observing the formation of a dark orange colour within one minute. After 1 h and 15 min stirring at room temperature, the reaction mixture was quenched by pouring into saturated NaHCO₃ solution (200 ml), bringing the total volume to 800 ml with water (resulting pH: 9.5) and maintaining stirring overnight. The resulting solution was ultrafiltered under a hood and concentrated in a rotary evaporator. A small portion was freeze-dried (0.15 g) for NMR analysis: total mesylate DS 5% mol/mol by proton NMR.

### Example 15

### Preparation of 6-O-p-toluenesulfonylhyaluronic Acid

A solution of HA:TBA salt (1.018 g; 1.64 mmol) (MW 20000) in 30 ml of dry DMF was treated with Et₃N (3.2 mL; 23.0 mmol) and TsCl (2.24 g; 11.7 mmol) at room temperature; the reaction mixture turned orange-red and the solution became viscous. After 1 hour, 6 ml of the reaction mixture was concentrated to half volume in a rotary evaporator and the sample was precipitated with acetone. A little amount of solid was dissolved in DMSO-d₆ and ¹H NMR and DOSY NMR spectra were obtained, which showed that the DS of the tosyl group was 16% mol/mol; 95 mg of the formylated sample were recovered.

¹H NMR (d₆-DMSO) ppm: 1.95 (s, 3H, NHCOCH₃), 2.45 (s, 0.49H, tosylate CH₃), 3.0÷5.4 (m, 12.3H, HA chain and anomeric), 7.5 (d, 0.34H, tosylate aromatics), 7.85 (d, 0.30H, tosylate aromatics), 8.0÷8.5 (m, 2.14H, O—CHO formyl ester groups).

The rest of the reaction was heated to 50° C. for a further hour, quenched in a saturated NaHCO₃ solution at pH 9, stirred for 24 hours, neutralised and filtered to remove solids. Than the solution was ultrafiltered and freeze-dried. ¹H NMR and DOSY NMR spectra in DMSO-d₆ were obtained, which showed that the DS of the tosyl group was 12% mol/mol. 55 mg of sample were recovered.

### Example 16

### Preparation of 6-O-p-toluenesulfonylhyaluronic Acid

A solution of HA:TBA salt (1.053 g; 1.70 mmol) (MW 20000) in 30 ml of dry DMF was treated with Et₃N (3.2 mL; 23.0 mmol) and TsCl (2.24 g; 11.7 mmol) at 0° C.; the reaction mixture turned orange-red and the solution became viscous. After 30 minutes, It was then brought to room temperature and after a further hour, the reaction mixture was concentrated to half volume in a rotary evaporator and the sample was precipitated with acetone. A little amount of solid was dissolved in DMSO-d₆ and ¹H NMR and DOSY NMR spectra were obtained, which showed that the DS of the tosyl group was 45% mol/mol; 700 mg of the formylated sample were recovered.

¹H NMR (d₆-DMSO) ppm: 1.95 (s, 3H, NHCOCH₃), 2.45 (s, 1.36H, tosylate CH₃), 3.0÷5.4 (m, 13.0H, HA chain and anomeric), 7.5 (d, 0.97H, tosylate aromatics), 7.85 (d, 0.90H, tosylate aromatics), 8.0÷8.5 (m, 2.33H, O—CHO formyl ester groups)

### Example 17

### Preparation of 6-O-Methanesulfonylhyaluronic Acid (HA-Ms)

To a solution of 500 mg (0.806 mmol) of TBA salt of HA (MW 20,000) in 20 ml of DMSO were added 829 μL (4.84 mmol) of DIEA by stirring under nitrogen. MsCl (188 μL; 2.42 mmol) was then added dropwise at room temperature, whereupon an orange solution was formed. After 1 h stirring at room temperature, the reaction mixture was quenched by pouring into saturated NaHCO₃ solution (40 ml), bringing the total volume to 100 ml with water (resulting pH: 9.5) and maintaining stirring overnight. The resulting solution was ultrafiltered under a hood and concentrated in a rotary evaporator. The solution was freeze-dried to afford 329 mg of a white solid. Total mesylate DS 77% mol/mol by proton NMR, primary mesylates 59% mol/mol by carbon NMR, selectivity 77% for the C6 position.

### Example 18

### Preparation of 6-O-Methanesulfonylhvaluronic Acid (HA-Ms)

To a solution of 500 mg (0.806 mmol) of TBA salt of HA (MW 20,000) in 20 ml of DMSO were added 414 μL (2.42 mmol) of DIEA by stirring under nitrogen. MsCl (94 μL; 1.21 mmol) was then added dropwise at room temperature, whereupon an orange solution was formed. After 1 h stirring at room temperature, the reaction mixture was quenched by pouring into saturated NaHCO₃ solution (40 ml), bringing the total volume to 100 ml with water (resulting pH: 9.5) and maintaining stirring overnight. The resulting solution was ultrafiltered under a hood and concentrated in a rotary evaporator. The solution was freeze-dried to afford 310 mg of a white solid. Total mesylate DS 34% mol/mol by proton NMR, primary mesylates 22% mol/mol by carbon NMR, selectivity 65% for the C6 position.

### Example 19

### Preparation of 6-O-Methanesulfonylhyaluronic Acid (HA-Ms)

To a solution of 500 mg (0.806 mmol) of TBA salt of HA (MW 20,000) in 20 ml of DMF were added 829 μL (4.84 mmol) of DIEA by stirring under nitrogen. MsCl (188 μL; 2.42 mmol) was then added dropwise at room temperature, whereupon a yellow solution was formed. After 1 h stirring at room temperature, the reaction mixture was quenched by adding saturated NaHCO₃ solution (40 ml) and bringing the total volume to 100 ml with water (resulting pH: 9.5); stirring was maintained overnight. The pH was raised to 10 and the suspension was stirred for 3 days, whereupon most of the solids dissolved. Then it was filtered and the resulting solution was ultrafiltered and concentrated in a rotary evaporator. The solution was freeze-dried to afford 277 mg of a white solid. Total mesylate DS 42% mol/mol by proton NMR, primary mesylates 40% mol/mol by carbon NMR, selectivity 95% for the C6 position.

### Example 20

### Preparation of 6-O-Methanesulfonylhvaluronic Acid (HA-Ms)

To a solution of 500 mg (0.806 mmol) of TBA salt of HA (MW 20,000) in 20 ml of DMF were added 829 μL (4.84 mmol) of DIEA by stirring under nitrogen at −10° C. MsCl (188 μL; 2.42 mmol) was then added dropwise and the resulting mixture was stirred for 1 h at −10° C. The reaction mixture was quenched by adding saturated NaHCO₃ solution (40 ml) and bringing the total volume to 100 ml with water (resulting pH: 9.5); stirring was maintained overnight. The resulting solution was ultrafiltered and concentrated in a rotary evaporator. The solution was freeze-dried to afford 207 mg of a white solid. Total mesylate DS 37% mol/mol by proton NMR, primary mesylates 37% mol/mol by carbon NMR, selectivity 100% for the C6 position.

### Example 21

### Preparation of 6-O-Methanesulfonylhyaluronic Acid (HA-Ms)

To a solution of 500 mg (0.806 mmol) of TBA salt of HA (MW 20,000) in 20 ml of N-methyl-2-pyrrolidone were added 829 μL (4.84 mmol) of DIEA by stirring under nitrogen. MsCl (188 μL; 2.42 mmol) was then added dropwise at room temperature, whereupon a yellow solution was formed. After 1 h stirring at room temperature, the reaction mixture was quenched by adding saturated NaHCO₃ solution (40 ml) and bringing the total volume to 100 ml with water (resulting pH: 9.5); stirring was maintained overnight. The resulting solution was ultrafiltered and concentrated in a rotary evaporator. The solution was freeze-dried to afford 310 mg of a white solid. Total mesylate DS 50% mol/mol by proton NMR, primary mesylates 31% mol/mol by carbon NMR, selectivity 62% for the C6 position.

### Example 22

### Preparation of 6-O-Methanesulfonylhyaluronic Acid (HA-Ms)

To a solution of 500 mg (0.806 mmol) of TBA salt of HA (MW 20,000) in 20 ml of N-methyl-2-pyrrolidone were added 829 μL (4.84 mmol) of DIEA by stirring under nitrogen at −10° C. MsCl (188 μL; 2.42 mmol) was then added dropwise and the resulting mixture was stirred for 1 h at −10° C. The reaction mixture was quenched by adding saturated NaHCO₃ solution (40 ml) and bringing the total volume to 100 ml with water (resulting pH: 9.5); stirring was maintained overnight. The resulting solution was ultrafiltered and concentrated in a rotary evaporator. The solution was freeze-dried to afford 250 mg of a white solid. Total mesylate DS 41% mol/mol by proton NMR, primary mesylates 39% mol/mol by carbon NMR, selectivity 95% for the C6 position. An HSQC NMR spectrum confirmed the selectivity.

### Example 23

### Preparation of 6-O-Methanesulfonylhyaluronic Acid (HA-Ms)

To a solution of 500 mg (0.806 mmol) of TBA salt of HA (MW 20,000) in 20 ml of N-methyl-2-pyrrolidone were added 829 μL (4.84 mmol) of DIEA by stirring under nitrogen at 0° C. MsCl (188 μL; 2.42 mmol) was then added dropwise and the resulting mixture was stirred for 1 h at 0° C. The reaction mixture was quenched by adding saturated NaHCO₃ solution (40 ml) and bringing the total volume to 100 ml with water (resulting pH: 9.5); stirring was maintained overnight. The resulting solution was ultrafiltered and concentrated in a rotary evaporator. The solution was freeze-dried to afford 275 mg of white solid. Total mesylate DS 44% mol/mol by proton NMR, primary mesylates 40% mol/mol by carbon NMR, selectivity 90% for the C6 position.

### Example 24

### Preparation of 6-O-Methotrexylhyaluronic Acid

A solution of HA-OMs:TBA salt from Example 10 (500 mg; 0.73 mmol) in DMSO (15 ml) was treated with a solution of methotrexate (833 mg; 1.83 mmol) in DMSO (10 ml) in the presence of solid cesium carbonate (596 mg; 1.83 mmol). The mixture was stirred under nitrogen at 80° C. for 18 h, whereupon it darkened with formation of solids. It was then cooled to ambient temperature, poured into 100 ml of water (pH 6.5), treated with 15 ml of saturated NaCl solution, and stirred for 1.5 h. Then solids were filtered off and the solution was ultrafiltered, concentrated and freeze-dried to give 131 mg of a yellow-brownish solid. DS of MTX by NMR: 40% mol/mol; ¹³C NMR shows that 40% of C6 is modified. HPLC analysis gave 32% w/w, corresponding to 40% mol/mol. In addition, the NMR revealed the absence of any residual secondary mesylate group and that the basic structure of HA was unchanged, except some of the C-6 position because of the substitution by MTX. This demonstrates that any possible leaving (mesylate) groups introduced at the secondary positions (C-4, C-2′, C-3′) during the mesylation reaction have been hydrolysed during the displacement reaction under the basic conditions either directly or by way of 2′,3′-anhydride formation followed by hydrolysis with the retention of configuration at those positions. The NMR spectrum was repeated on the same sample after 3-months storage at room temperature, it provides the same peaks and the same intensity as those obtained on the freshly prepared product, thus indicating that the substitution degree is maintained and no by-products are formed.

### Example 25

### Preparation of HA-Cl:TBA Salt

50 g of hyaluronan sodium salt were suspended in 900 mL of dry dimethylformamide under nitrogen, with mechanical stirring at 20° C. The suspension was then cooled to −10° C. and 97 mL of methanesulfonyl chloride were added during 30 min. After additional 30 min at −10° C., the temperature was raised to 20° C. After 1 h the temperature was gradually raised (during 1 h) to 60° C. and stirring was continued for 18 h. The reaction mixture was then poured in portions into a mixture of ice and sodium carbonate solution (4 L, initial pH=11) with vigorous mixing, maintaining the pH around 9 by addition of 1.5 M NaOH when required. The resulting brownish suspension (final volume 6 L) was stirred at pH 9.5 at room temperature for about 48 h, whereupon a clear solution formed. This was filtered to remove solids and then ultrafiltered (10 KDa cut-off membrane). The resulting solution was concentrated in a rotary evaporator to a final volume of about 1 litre and treated with amberlite IRA-120 loaded with TBA. Then it was freeze-dried to afford 46.7 g of HA-6-CI: TBA salt as an off-white solid (DS 64% mol/mol, determined by ¹³C NMR).

### Example 26

### Preparation of Ha-Ibuprofen

HA-Ms:TBA salt (400 mg; 0.64 mmol) as prepared in example 12 and ibuprofen (333 mg; 1.61 mmol) were dissolved in DMSO (16 ml) by stirring under nitrogen at room temperature. Solid cesium carbonate (264 mg; 0.81 mmol) was added and the suspension was heated at 70° C. for 20 h with stirring. The resulting yellow-orange solution was poured into 150 ml of water (pH was 6.5) and 10 ml of saturated NaCl solution were added. After stirring for 30 min, the solution was ultrafiltered, concentrated and freeze-dried to give 0.15 g of a white solid. DS by proton NMR: 27% mol/mol.

### Example 27

### Preparation of Ha-Ibuprofen

HA-Cl:TBA salt (1 g; 1.6 mmol) as prepared in example 25 and ibuprofen (670 mg; 3.2 mmol) were dissolved in DMSO (50 ml) by stirring under nitrogen at room temperature. Solid cesium carbonate (264 mg; 0.81 mmol) was added and the suspension was heated at 80° C. for 40 h with stirring. The resulting dark yellow solution was poured into 100 ml of water (pH was 8) and then ultrafiltered, concentrated and freeze-dried to give g of a light brown solid. DS by proton NMR: 20% mol/mol.

### Example 28

### Preparation of Ha-Penicillin G

A solution of HA-Ms:TBA salt (400 mg; 0.64 mmol) as prepared in example 12, 18-crown-6 (338 mg; 1.28 mmol) and Penicillin G sodium salt (574 mg; 1.61 mmol) in DMSO (16 ml) was heated at 70° C. for 20 h with stirring.

The resulting yellow solution was poured into 150 ml of water (pH was 6.5) and 10 ml of saturated NaCl solution were added. After stirring for 30 min, the solution was ultrafiltered, concentrated and freeze-dried to give 0.29 g of a white solid. DS by proton NMR: 26% mol/mol.

### Example 29

### Preparation of Ha-Penicillin G

HA-Cl:TBA salt (1 g; 1.6 mmol) as prepared in example 25, 18-crown-6 (840 mg; 3.2 mmol) and Penicillin G sodium salt (1.13 g; 3.2 mmol) were dissolved in DMSO (50 ml) by stirring at room temperature. The solution was heated at 80° C. for 40 h with stirring, then it was poured into 100 ml of water (pH was 7.4) and ultrafiltered, concentrated and freeze-dried to give 1 g (yield 64%) of a pale yellow solid. DS by proton NMR: 6% mol/mol.

### Example 30

### Preparation of Ha-Albumin

HA-Cl:TBA salt (1 g; 1.6 mmol) as prepared in example 25 and Human serum Albumin (300 mg) were dissolved in DMSO (50 ml) by stirring under nitrogen at room temperature. Solid cesium carbonate (264 mg; 0.81 mmol) was added and the suspension was heated at 80° C. for 40 h with stirring. The resulting brown solution was poured into 100 ml of water (pH was 9.5) and then ultrafiltered, concentrated and freeze-dried to give 0.9 g of a light brown solid. DS by HPLC RP: 5% mol/mol.

### Example 31

### Preparation of 6-O-Methotrexylhyaluronic Acid

HA:TBA salt (250 mg; 0.403 mmol; MW 20,000) was dissolved in DMSO (10 ml) by stirring and gentle heating under nitrogen; triethylamine (452 μL; 3.22 mmol) was then added at room temperature followed by dropwise addition of MsCl (157 μL; 2.02 mmol), whereupon a yellow solution formed. After 1 h stirring at room temperature, further 0.50 ml of triethylamine were added, the reaction flask was connected to the vacuum and gently heated up to 50° C. (bath temperature), until gas evolution ceased. Then 1.10 g (2.43 mmol) of methotrexate and 792 mg (2.43 mmol) of cesium carbonate were added and the mixture was stirred at 80° C. overnight. Half of the reaction mixture was quenched by pouring into water (20 ml); pH 6.3. The pH was adjusted to 6.8 with saturated NaHCO₃ solution and then 10 ml of saturated NaCl solution were added. After stirring for 10 min, the solution was ultrafiltered, concentrated in a rotary evaporator and freeze-dried to give 60 mg of a yellow solid. The DS of MTX was found to be 11.6% w/w by HPLC and was confirmed by NMR analysis (12% mol/mol), which also showed a small percentage of left mesylates on the polymer.

The rest of the reaction mixture was worked up after further 24 h at 80° C. (40 h overall) as described above, to afford 103 mg of a yellow solid. DS in MTX 14.4% w/w by HPLC, confirmed by NMR analysis (15% mol/mol), which did not show any mesylate left on the polymer. MW 269610, PI 10.5. Cross-linking ester bonds were cleaved by hydrolyzing the freeze dried product in 10 ml of a carbonate buffer (pH 10) for 8 h. After neutralization and dialysis, freeze-drying afforded 96 mg of a yellow solid. The DS of MTX in the product was 12.6% w/w by HPLC, which was confirmed by NMR analysis (13% mol/mol); MW 27,120, PI 1.9.

### Example 32

### Preparation of 6-O-methotrexylhyaluronic Acid

To a solution of HATBA (50 g, Mw 70,000) in 1000 ml of dry DMF under stirring, mesylchloride (10 eq) was added dropwise in 1 hr time at −10° C. under N₂ flow. The mixture was maintained for 1 hour at room temperature and then heated at 60° C. for 16 hr. The work-up allow the obtainment of 46.9 g of HA-CL having chlorine content 4.2% w/w (¹³C-NMR).

A solution of the HA-Cl TBA salt (20 g) in DMSO (1.25 L) is treated with MTX (29.3 g) and cesium carbonate (21 g) at 80° C. for 40 h, giving 5.6 g of a yellow solid.

¹³C-NMR spectrum confirmed the occurrence of the linkage in position 6 of N-acetyl-D-glucosamine: the peak at 64 ppm is assigned at CH₂O-MTX and its intensity corresponds to the decrease of the peak at 44 ppm (CH₂Cl) compared to the parent chlorine derivative. The MTX content was 18.8% w/w (HPLC); free MTX was 0.1% w/w, water content: 8.2% w/w; MW: 11.000; PI: 1.4. In addition, the NMR reveals the presence of residual chlorine which amounts to 1.76% w/w.

### Example 33

### Preparation of 6-O-Methanesulfonylhvaluronic Acid TBA Salt (HA-Ms:TBA)

To a solution of 5.0 g (8.1 mmol) of HA:TBA (MW 5,000) in 100 mL of dry DMF were added 3.1 mL of DIEA (5.62 mL, 33.9 mmol) under stirring and N₂ flow at −10° C. MsCl (1.25 mL; 16.1 mmol) was then added dropwise and the resulting mixture was stirred for 1 h at −10° C. The reaction mixture was quenched by adding saturated Na₂CO₃ solution (200 mL) and bringing the total volume to 1 L with water; pH was adjusted to 10.5 with dilute HCl solution and stirring was maintained overnight. The resulting solution was ultrafiltered and concentrated in a rotary evaporator. A small portion was freeze-dried (100 mg) for NMR analysis: primary mesylates 30% mol/mol by NMR, selectivity 100% for C6. (3.2 g; Mw: 6,925; P.I. 1.87)

### Example 34

### Preparation of 6-O-Methanesulfonylhvaluronic Acid TBA Salt (HA-Ms:TBA)

To a solution of 10.0 g (16.1 mmol) of TBA salt of HA (MW 20,000) in 250 ml of DMF were added 7.58 ml (44.3 mmol) of DIEA by stirring under nitrogen at −10° C. MsCl (1.56 mL; 20.1 mmol) was then added dropwise and the resulting mixture was stirred for 1 h at −10° C. The reaction mixture was quenched by adding saturated Na₂CO₃ solution (400 mL) and bringing the total volume to 2 L with water; pH was adjusted to 10.5 with dilute HCl solution and stirring was maintained overnight. The resulting solution was ultrafiltered and concentrated in a rotary evaporator. A small portion was freeze-dried (100 mg) for NMR analysis: primary mesylates 24% mol/mol by NMR, selectivity 100% for C6.

The rest of the solution was treated with amberlite IRA-120 loaded with TBA and freeze-dried to afford 9.92 g of a white solid (HA-Ms:TBA salt).

### Example 35

### Preparation of Ha-Cl:Sodium Salt

5 g of hyaluronan sodium salt (MW 200,000) were suspended in 90 mL of dry dimethylformamide under nitrogen, with mechanical stirring at 20° C. The suspension was then cooled to −10° C. and 9.7 mL of methanesulfonyl chloride were added during 30 min. After additional 30 min at −10° C., the temperature was raised to 20° C. After 1 h the temperature was gradually raised (during 1 h) to 60° C. and stirring was continued for 18 h. The reaction mixture was then poured in portions into a mixture of ice and sodium carbonate solution (400 mL, initial pH=11) with vigorous mixing, maintaining the pH around 9 by addition of 1.5 M NaOH when required. The resulting brownish suspension (final volume 500 mL) was stirred at pH 9.5 at room temperature for about 48 h, whereupon a clear solution formed. This was filtered to remove solids and then ultrafiltered (10 KDa cut-off membrane). The resulting solution was concentrated and freeze-dried to afford 4.05 g of HA-6-CI: sodium salt as an off-white solid (DS 17% mol/mol, determined by ¹³C NMR). MW 79,560, P.I. 3.5.

### Example 36

### Preparation of Ha-Cl: Sodium Salt

5 g of hyaluronan sodium salt (MW 500,000) were suspended in 90 mL of dry dimethylformamide under nitrogen, with mechanical stirring at 20° C. The suspension was then cooled to −10° C. and 9.7 mL of methanesulfonyl chloride were added during 30 min. After additional 30 min at −10° C., the temperature was raised to 20° C. After 1 h the temperature was gradually raised (during 1 h) to 70° C. and stirring was continued for 21 h. The reaction mixture was then poured in portions into a mixture of ice and sodium carbonate solution (400 mL, initial pH=11) with vigorous mixing, maintaining the pH around 9 by addition of 1.5 M NaOH when required. The resulting brownish suspension (final volume 500 mL) was stirred at pH 9.5 at room temperature for about 48 h, whereupon a clear solution formed. This was filtered to remove solids and then ultrafiltered (10 KDa cut-off membrane). The resulting solution was concentrated and freeze-dried to afford 3.56 g of HA-6-Cl: sodium salt as an off-white solid (DS 10% mol/mol, determined by ¹³C NMR). MW 53,830, P.I. 4.02.

### Example 37

### Preparation of 6-O-Methanesulfonylhvaluronic Acid TBA Salt (HA-Ms:TBA)

To a solution of 10.0 g (16.1 mmol) of TBA salt of HA (MW 180,000) in 500 ml of DMF were added 9.16 ml (53.4 mmol) of DIEA by stirring under nitrogen at −10° C. MsCl (1.87 mL; 24.2 mmol) was then added dropwise and the resulting mixture was stirred for 1 h at −10° C. The reaction mixture was quenched by adding saturated Na₂CO₃ solution (400 mL) and bringing the total volume to 2 L with water; pH was adjusted to 10.5 with dilute HCl solution and stirring was maintained overnight. The resulting solution was ultrafiltered and concentrated in a rotary evaporator. A small portion was freeze-dried (150 mg) for NMR analysis: primary mesylates 30% mol/mol by NMR, selectivity 100% for C6.

The rest of the solution was treated with amberlite IRA-120 loaded with TBA and freeze-dried to afford 9.80 g of a white solid (HA-Ms:TBA salt).

### Example 38

### Preparation of 6-O-Methanesulfonylhvaluronic Acid TBA Salt (HA-Ms:TBA)

To a solution of 20.0 g (32.2 mmol) of TBA salt of HA (MW 180,000) in 1000 ml of DMF were added 36.7 ml (214 mmol) of DIEA by stirring under nitrogen at −10° C. MsCl (7.48 μL; 97 mmol) was then added dropwise and the resulting mixture was stirred for 1 h at −10° C. The reaction mixture was quenched by adding saturated Na₂CO₃ solution (800 mL) and bringing the total volume to 4 L with water; pH was adjusted to 10.5 with dilute HCl solution and stirring was maintained overnight. The resulting solution was ultrafiltered and concentrated in a rotary evaporator. A small portion was freeze-dried (120 mg) for NMR analysis: primary mesylates 55% mol/mol by NMR, selectivity 100% for C6.

The rest of the solution was treated with amberlite IRA-120 loaded with TBA and freeze-dried to afford 19.95 g of a white solid (HA-Ms:TBA salt).

### Example 39

### Preparation of 6-O-Methanesulfonylhvaluronic Acid TBA Salt (HA-Ms:TBA)

To a solution of 2.00 g (3.22 mmol) of TBA salt of HA (MW 500,000) in 150 ml of DMF were added 3.67 ml (21.4 mmol) of DIEA by stirring under nitrogen at −10° C. MsCl (750 μL; 9.7 mmol) was then added dropwise and the resulting mixture was stirred for 1 h at −10° C. The reaction mixture was quenched by adding saturated Na₂CO₃ solution (80 mL) and bringing the total volume to 1 L with water; pH was adjusted to 10.5 with dilute HCl solution and stirring was maintained overnight. The resulting solution was ultrafiltered and concentrated in a rotary evaporator. A small portion was freeze-dried (90 mg) for NMR analysis: primary mesylates 70% mol/mol by NMR, selectivity 100% for C6.

The rest of the solution was treated with amberlite IRA-120 loaded with TBA and freeze-dried to afford 1.94 g of a white solid (HA-Ms:TBA salt).

### Example 40

### Preparation of 6-O-Methotrexylhyaluronic Acid

A solution of HA-OMs:TBA salt from Example 34 (8.0 g; 12.9 mmol) in DMSO (250 ml) was treated with a solution of methotrexate (14.66 g; 32.3 mmol) in DMSO (150 ml) in the presence of solid cesium carbonate (10.5 g; 32.2 mmol). The mixture was stirred under nitrogen at 80° C. for 20 h. It was then cooled to ambient temperature and poured into a carbonate buffer, adjusting the pH to 9.7 and the volume to 2 L. After stirring for 18 h the solution was neutralized, filtered, ultrafiltered, concentrated and freeze-dried to give 4.70 g of a yellow solid. DS of MTX by NMR: 7.5% mol/mol; MW 27,960, P.I. 1.92.

### Example 41

### Preparation of 6-O-Methotrexylhyaluronic Acid

A solution of HA-OMs:TBA salt from Example 37 (7.0 g; 11.3 mmol) in DMSO (670 ml) was treated with a solution of methotrexate (12.79 g; 28.1 mmol) in DMSO (120 ml) in the presence of solid cesium carbonate (9.15 g; 28.1 mmol). The mixture was stirred under nitrogen at 75° C. for 18 h. It was then cooled to ambient temperature and poured into a carbonate buffer, adjusting the pH to 8.8 and the volume to 2.5 L. After stirring for 24 h the solution was neutralized, filtered, ultrafiltered, concentrated and freeze-dried to give 4.30 g of a yellow solid. DS of MTX by NMR: 13% mol/mol; MW 208,400, P.I. 2.18.

### Example 42

### Preparation of 6-O-Methotrexylhyaluronic Acid

A solution of HA-OMs:TBA salt from Example 38 (13.2 g; 21.3 mmol) in DMSO (1270 ml) was treated with a solution of methotrexate (24.13 g; 53.1 mmol) in DMSO (120 ml) in the presence of solid cesium carbonate (17.26 g; 53.1 mmol). The mixture was stirred under nitrogen at 75° C. for 18 h. It was then cooled to ambient temperature and poured into a carbonate buffer, adjusting the pH to 10.0 and the volume to 5 L. After stirring for 18 h the solution was neutralized, filtered, ultrafiltered, concentrated and freeze-dried to give 6.52 g of a yellow solid. DS of MTX by NMR: 20% mol/mol; MW 217,300, P.I. 2.02.

### Example 43

### Preparation of 6-O-Methotrexylhyaluronic Acid

A solution of HA-OMs:TBA salt from Example 39 (500 mg; 0.74 mmol) in DMSO (80 ml) was treated with a solution of methotrexate (1.01 g; 2.23 mmol) in DMSO (10 ml) in the presence of solid cesium carbonate (726 mg; 2.23 mmol). The mixture was stirred under nitrogen at 80° C. for 22 h. It was then cooled to ambient temperature and poured into a carbonate buffer, adjusting the pH to 10.0 and the volume to 400 mL. After stirring for 18 h the solution was neutralized, filtered, ultrafiltered, concentrated and freeze-dried to give 260 mg of a yellow solid. DS of MTX by NMR: 11% mol/mol; MW 460,100, P.I. 2.21.

### Example 44

### Preparation of 6-O-Methotrexylhyaluronic Acid

A solution of HA-OMs sodium salt from Example 33 (1.0 g; 2.0 mmol) in DMSO (40 ml) was treated with a solution of methotrexate (1.83 g; 4 mmol) in DMSO (40 ml) in the presence of solid cesium carbonate (1.30; 2 mmol). The mixture was stirred under nitrogen at 80° C. for 20 h. The solution was neutralized using Na₂CO₃ saturated solution bringing the final volume to 500 mL, filtered, ultrafiltered, concentrated and freeze-dried to give 500 mg of a yellow solid. DS of MTX by HPLC: 7.8% w/w; MW 16,000, P.I. 2.4.

### Example 45

### Preparation of Ha-Ibuprofen

HA-Ms:TBA salt (500 mg; 0.80 mmol) as prepared in example 20 and ibuprofen (416 mg; 2.01 mmol) were dissolved in DMSO (20 ml) by stirring under nitrogen at room temperature. Solid cesium carbonate (330 mg; 1.01 mmol) was added and the suspension was heated at 70° C. for 20 h with stirring. The resulting solution was poured into 200 ml of water (pH was 6.5) and 10 ml of saturated NaCl solution were added. After stirring for 30 min, the solution was ultrafiltered, concentrated and freeze-dried to give 0.22 g of a white solid. DS by proton NMR: 30% mol/mol.

### Example 46

### Preparation of Ha-Naproxen

HA-Ms:TBA salt (500 mg; 0.80 mmol) as prepared in example 20 and naproxen (463 mg; 2.01 mmol) were dissolved in DMSO (20 ml) by stirring under nitrogen at room temperature. Solid cesium carbonate (330 mg; 1.01 mmol) was added and the suspension was heated at 70° C. for 20 h with stirring. The resulting solution was poured into 200 ml of water (pH was 6.6) and 10 ml of saturated NaCl solution were added. After stirring for 30 min, the solution was ultrafiltered, concentrated and freeze-dried to give 0.27 g of a white solid. DS by proton NMR: 28% mol/mol.

### Example 47

### Preparation of Ha-Lisinopril

HA-Ms:TBA salt (500 mg; 0.80 mmol) as prepared in example 20 and lisinopril (887 mg; 2.01 mmol) were dissolved in DMSO (25 ml) by stirring under nitrogen at room temperature. Solid cesium carbonate (655 mg; 2.01 mmol) was added and the suspension was heated at 70° C. for 20 h with stirring. The resulting solution was poured into 200 ml of water (pH was 6.5) and 10 ml of saturated NaCl solution were added. After stirring for 30 min, the mixture was filtered, ultrafiltered, concentrated and freeze-dried to give 0.20 g of a white solid. DS by proton NMR: 26% mol/mol.

### Example 48

### Preparation of Ha-Nalidixate

HA-Ms:TBA salt (500 mg; 0.80 mmol) as prepared in example 20 and nalidixic acid

(467 mg; 2.01 mmol) were dissolved in DMSO (20 ml) by stirring under nitrogen at room temperature. Solid cesium carbonate (330 mg; 1.01 mmol) was added and the suspension was heated at 70° C. for 20 h with stirring. The resulting solution was poured into 200 ml of water (pH was 6.6) and 10 ml of saturated NaCl solution were added. After stirring for 30 min, the solution was ultrafiltered, concentrated and freeze-dried to give 0.26 g of a white solid. DS by proton NMR: 30% mol/mol.

### Example 49

### Preparation of Ha-Penicillin G

A solution of HA-Ms:TBA salt (400 mg; 0.64 mmol) as prepared in example 20, 18-crown-6 (338 mg; 1.28 mmol) and Penicillin G sodium salt (574 mg; 1.61 mmol) in DMSO (16 ml) was heated at 70° C. for 20 h with stirring.

The resulting yellow solution was poured into 150 ml of water (pH was 6.5) and 10 ml of saturated NaCl solution were added. After stirring for 30 min, the solution was ultrafiltered, concentrated and freeze-dried to give 0.27 g of a white solid. DS by proton NMR: 31% mol/mol.

### Example 50

### Preparation of Ha-Cefazolin

A solution of HA-Ms:TBA salt (400 mg; 0.64 mmol) as prepared in example 20, 18-crown-6 (338 mg; 1.28 mmol) and cefazolin sodium salt (767 mg; 1.61 mmol) in DMSO (18 ml) was heated at 70° C. for 20 h with stirring.

The resulting solution was poured into 180 ml of water (pH was 6.7) and 10 ml of saturated NaCl solution were added. After stirring for 30 min, the solution was ultrafiltered, concentrated and freeze-dried to give 0.25 g of a white solid. DS by proton NMR: 29% mol/mol.

1. A drug delivery system comprising hyaluronic acid and a therapeutic active agent, whereby the active agent is covalently linked at the C-6 position of the 1V-acetyl-D-glucosamine residue of the hyaluronic acid with the exception of active agents of formula (I) 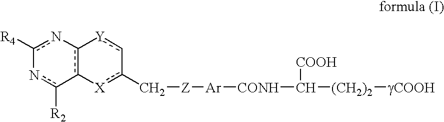
wherein:
R₂ and R₄ independent from one another represent —NH₂, —OH, —OCH₃, C₁-C₅ alkyl, or ═O; X and Y represent —C(R₅)═, —CH(R₅)—, or —NH—, —N═), wherein R₅ represents —H or C₁-C₅ alkyl; Z represents —CH(R₁₀)—, —N(R₁₀)—, or —O—; R₁₀ represents —H, C₁-C₅ alkyl, C₁-C₅ alkenyl, C₁-C₅ alkynyl, or 5-6 membered heterocyclic ring with 1-3 heteroatoms selected in the group consisting of nitrogen, sulphur and oxygen; Ar represents 1,4-phenyl group, 1,4-phenyl group condensed with one or more 5-6 membered aromatic rings, or 1,4-phenyl group condensed with one or more 5-6 membered heterocycles, wherein said Ar is optionally substituted with R₂; rings A and b, independently from one another, may be aromatic or non-aromatic, where in said delivery system no other hydroxyl groups of hyaluronic acid are involved in the chemical linkage with the drug.

2. The drug delivery system of claim 1 wherein the linkage between the hyaluronic acid and the active agent is an ester, an amino, an ether, a thioether, or an amide.

3. The drug delivery system of claim 1 wherein the therapeutic active agent is selected from the group consisting of analgesic, antihypertensive, anesthetic, diuretic, bronchodilator, calcium channel blocker, cholinergic, estrogen, immunomodulator, immunosuppressant, lipotropic, anxiolytic, antiulcerative, antiarrhytmic, antianginal, antibiotic, anti-inflammatory, antiviral, thrombolytic, vasodilator, antipyretic, antidepressant, antipsychotic, antitumour, mucolytic, narcotic antagonist, anticonvulsant, antihistaminic, antifungal, and antipsoriatic therapeutic active agents.

4. The drug delivery system of claim 1 wherein the active agent is present in amount between 0.1 and 60% w/w with respect to the total weight of the drug delivery system (preferably 1 and 50%).

5. The drug delivery system of claim 1 wherein the secondary hydroxyl groups of the hyaluronic acid are derivatised to form a group selected from —OR, —OCOR, —SO₂H, —OPO₃H₂, —O—CO—(CH₂)ₙ—COOH, or —O—(CH₂)ₙ—OCOR, wherein n is 1-4 and R is C₁-C₁₀ alkyl, —NH₂, or —NHCOCH₃.

6. The drug delivery system of claim 1 in the acid form or salified with alkaline metals, earth-alkaline metals, transition metals.

7. (canceled)

8. A pharmaceutical composition comprising the drug delivery system of claim 1 in admixture with a pharmaceutically acceptable excipient and/or diluent.

9. The pharmaceutical composition of claim 8 in injectable form.

10. Process for the preparation of a drug delivery system comprising:
(a) introducing a sulfonate group at the C-6 position of the N-acetyl-D-glucosamine units of a hyaluronic acid in the salt form thus obtaining a HA-6-sulfonated;
(b) forming a chemical linkage between the C6 position of the HA-6-sulfonated and a therapeutic active agent by displacing the sulfonated group at the C6 position of hyaluronic acid with a nucleophilic group present on the therapeutic active agent, thereby obtaining a HA-6-active agent; and
(c) recovering the HA-6-active agent.

11. The process of claim 10 wherein the linkage between the hyaluronic acid and the active agent is an ester, an amino, an ether, a thioether, or an amide.

12. The process of claim 11 wherein the linkage between the hyaluronic acid and the active agent is an ester.

13. The process of claims claim 10 wherein the reagent for introducing the sulfonate group is an alkyl- or aryl-sulfonyl halide in the presence of an organic or inorganic base.

14. The process of claim 13 wherein the reagent is methylsulfonyl chloride or toluene-p-sulfonyl chloride and the organic base is diisopropylethylamine or triethylamine.

15. A drug delivery system comprising hyaluronic acid and a compound of formula (I), whereby the carboxylic group of compound of formula (I) is covalently linked at the C-6 position of the N-acetyl-D-glucosamine residue of the hyaluronic acid by means of an ester linkage 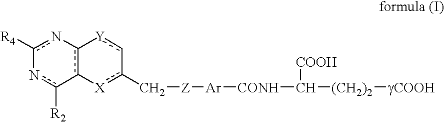
said drug delivery system being devoid of any residual leaving groups, both on the primary and secondary positions of the hyaluronic acid units, whereby said drug delivery system is obtained by a process, comprising:
(a) introducing at the C-6 position of the N-acetyl-D-glucosamine units of a hyaluronic acid in the free form or in the salt form a leaving group selected from the group consisting of sulfonate group, phosphonate group, cyanide, nitrite, sulphate group, halogensulfate group, nitrate, halogensulfite thus obtaining a HA-6-activated;
(b) forming an ester linkage between the C6 position of the HA-6-activated and the compound of formula (I) by displacing the leaving group at the C6 position of the hyaluronic acid with a carboxylic group present on compound (I), thereby obtaining a HA-6-compound of formula (I);
(c) recovering the HA-6-compound of formula (I).

16. The drug delivery system of claim 15 having a C6-DS_{w} comprised between 1 and 50%.

17. The drug delivery system of claim 15 wherein the compound of formula (I) is methotrexate.

18. The drug delivery system of claim 15 wherein the HA-6-activated obtained from step (a) is isolated from the reaction mixture and then reacted with the therapeutic active agent according to step (b).

19. The drug delivery system of claim 15 wherein step (b) is performed directly on the reaction mixture of step (a) containing the HA-6-activated.

20. The drug delivery system of claim 15 wherein the reagent used for introducing the sulfonate group is an alkyl- or aryl-sulfonyl halide in the presence of an organic or inorganic base, preferably organic.

21. The drug delivery system of claim 20 wherein the reagent is methylsulfonyl chloride or toluene-p-sulfonyl chloride and the organic base is diisopropylethylamine or triethylamine.

22. (canceled)

23. A pharmaceutical composition comprising the drug delivery system of claim 15 in admixture with a pharmaceutically acceptable excipient and/or diluent.

24. The pharmaceutical composition of claim 23 in injectable form.

25. HA-6-activated obtainable by introducing, at the C-6 position of the N-acetyl-D-glucosamine units of hyaluronic acid in the free form or in the salt form, a leaving group selected from the group consisting of sulfonate, phosphonate, cyanide, nitrite, sulphate, halogensulfate, nitrate, and halogensulfite.

26. HA-6-activated of claim 25, wherein the leaving group is sulphonate group.

27. HA-6-activated of claim 25 having a C6-DSₘₒₗ comprised between 10 and 91%.

28. HA-6-activated of claim 25 having a C6-DSₘₒₗ between 20 and 90%.

29. HA-6-activated of claim 25 having a C6-DSₘₒₗ between 40 and 80%.
